(19) United States
(12) Patent Application Publication
(10) Pub. No.: US 2024/0043932 A1
(43) Pub. Date: Feb. 8, 2024

(54) SYSTEMS AND METHODS TO OPTIMIZE AND FRACTIONIZE RADIATION FOR PERSONALIZED RADIATION THERAPY

(71) Applicants: H. Lee Moffitt Cancer Center and Research Institute, Inc., Tampa, FL (US); University of South Florida, Tampa, FL (US)

(72) Inventors: Javier Torres-Roca, St. Petersburg, FL (US); Jeffrey Peacock, Tampa, FL (US); Xiaoming Liu, Tampa, FL (US)

(21) Appl. No.: 17/641,221

(22) PCT Filed: Sep. 14, 2020
(86) PCT No.: PCT/US2020/050661

§ 371 Date: Mar. 8, 2022

Related U.S. Application Data
(60) Provisional application No. 62/899,973, filed on Sep. 13, 2019.

(51) Int. Cl. C12Q 1/6886; G16B 25/00; G16B 40/20; G16H 20/40

(52) U.S. Cl. CPC C12Q 1/6886; G16B 25/00; G16B 40/20; G16H 20/40; C12Q 2600/106; C12Q 2600/158

(57) ABSTRACT

Systems and methods for personalized dose and fractionation for radiation therapy are described herein. An example method can include predicting a patient-specific radiosensitivity parameter alpha (α) value for a tumor, where the patient-specific radiosensitivity parameter alpha (α) value is predicted based on a first set of signature genes. The method can also include predicting a patient-specific radiosensitivity parameter beta (β) value for the tumor, where the patient-specific radiosensitivity parameter beta (β) value is predicted based on a second set of signature genes. The method can further include calculating a patient-specific dose and fractionation using a radiation cytotoxicity score (RCS) function and the patient-specific radiosensitivity parameters alpha (α) and beta (β) values. RCS is predictive of clinical outcome.

### CROSS-REFERENCE TO RELATED APPLICATIONS

This application claims the benefit of U.S. provisional patent application No. 62/899,973, filed on Sep. 13, 2019, and titled “SYSTEMS AND METHODS TO OPTIMIZE AND FRACTIONIZE RADIATION FOR PERSONALIZED RADIATION THERAPY,” the disclosure of which is expressly incorporated herein by reference in its entirety.

### BACKGROUND

Radiation Therapy (RT) is a highly utilized, efficacious and cost-effective therapeutic option for cancer patients. Personalized RT holds the promise that the diagnosis, prevention, and treatment of cancer can be based on individual assessment of risk.

For example, RT is responsible for approximately 40% of all cancer cures yet it continues to follow the “one size fits all” approach. Previous work includes development of Radiation Sensitivity Index (RSI) and Genomic Adjusted Radiation Dosing (GARD) to usher radiation into the precision medicine era. RSI and GARD are described in WO2016/179422, published Nov. 10, 2016, titled “SYSTEMS AND METHODS FOR PROVIDING PERSONALIZED RADIATION THERAPY.” RSI was trained on the NCI60 to predict survival fraction 2 Gy (“SF2”) from genomic expression. GARD was developed based on the LQ model to derive patient specific α from RSI as a proxy for SF2. (See Scott J G, Berglund A, Schell M J, et al. A genome-based model for adjusting radiotherapy dose (GARD): a retrospective, cohort-based study. Lancet Oncol. 2017; 18(2):202-211. doi:10.1016/51470-2045(16)30648-9). GARD has been validated across multiple clinical cohorts as a continuous variable for predicting outcomes in patients that have received radiation.

The LQ model was used as a basis for GARD because it is the most widely accepted radiation response model used in the clinic today. It describes the surviving fraction (SF) of clonogenic cells as a function of radiation dose (D):

SF=e^{-αD-βD\hat{ }2}

The two parameters of this model, α and ß, represent radiosensitivity of the irradiated cells, α is linearly related to dose while β is quadratically related to dose. The ratio of the two parameters, α/β, is a measure of the fractionation sensitivity of the cells: cells with a lower α/β, are more sensitive fraction size. The LQ model has shown its clinical usefulness in predicting the sparing effect of fractionated radiotherapy, and in comparing equivalent doses of different fractionation schedules.

Historically, α and β values of the LQ model are the basis of most disease site dosing and fractionation schemas with the broad assumption of homogeneity across tumor tissue types (α/β=10). Significant evidence shows certain tumor types exhibit a much lower α/β ratio and may benefit from hypofractionated regimens. Treatment of prostate cancer is an excellent example of how utilizing a prostate specific α/β (^{˜}2-3) has changed standard of care. The estimation of radiotherapeutic outcome, and therapeutic window strongly depends on a reliable estimation of the LQ parameters α, β and α/β.

Many studies have estimated α, β and α/β in different histological tumor types either through cell lines or clinical radiotherapy data fit to tumor control probability. However, prior to RSI/GARD no study has estimated a based on genomic information.

### SUMMARY

Systems and methods for personalized dose and fractionation for radiation therapy are described herein.

An example method includes predicting a patient-specific radiosensitivity parameter alpha (α) value for a tumor, where the patient-specific radiosensitivity parameter alpha (α) value is predicted based on a first set of signature genes. The method also includes predicting a patient-specific radiosensitivity parameter beta (β) value for the tumor, where the patient-specific radiosensitivity parameter beta (β) value is predicted based on a second set of signature genes. The method further includes calculating a patient-specific dose and fractionation using a radiation cytotoxicity score (RCS) function and the patient-specific radiosensitivity parameters alpha (α) and beta (β) values. RCS is predictive of clinical outcome.

In some implementations, the method optionally includes administering radiation therapy to a subject based upon the calculated patient-specific dose and fractionation. Optionally, the administered radiation therapy is a hypo-fractionated radiation therapy regimen.

Alternatively or additionally, the patient-specific radiosensitivity parameter alpha (α) value is predicted using a first machine learning model. Alternatively or additionally, the patient-specific radiosensitivity parameter beta (β) value is predicted using a second machine learning model. Optionally, the first and second machine learning models are support vector machines (SVMs).

Alternatively or additionally, the first set of signature genes includes at least one of HTRA1, C5orf17, KLHL6, DUSP27, FOS, PLCB4, WT1, PFN2, GNAI1, EVA1C, PIK3CG, ST8SIA6-AS1, and ATP8B4.

Alternatively or additionally, the second set of signature genes includes at least one of RRAGD, C5orf17, SOX8, CHRNA9, UMODL1, HOXC10, FGFBP1, HEMGN or EDAG, WT1, SCG5, CRYAB, GPX1, ZBED2, MAP2, RHAG, MSLN, and HSPA2.

Alternatively or additionally, the RCS function is based on a linear quadratic model for cell survival.

Alternatively or additionally, the subject is predicted to have a favorable clinical outcome when an RCS value is greater than a threshold.

Alternatively or additionally, the patient-specific dose and fractionation comprises a fraction dose and a number of fractions.

Alternatively or additionally, the tumor is a rectal, lung, or breast tumor.

An example system for personalized radiation therapy is described herein. The system includes a processor, and a memory operably coupled to the processor, where the memory having computer-executable instructions stored thereon. The processor is configured to predict a patient-specific radiosensitivity parameter alpha (α) value for a tumor, where the patient-specific radiosensitivity parameter alpha (α) value is predicted based on a first set of signature genes. The processor is also configured to predict a patient-specific radiosensitivity parameter beta (β) value for the tumor, where the patient-specific radiosensitivity parameter beta (β) value is predicted based on a second set of signature genes. The processor is further configured to calculate a patient-specific dose and fractionation using a radiation cytotoxicity score (RCS) function and the patient-specific radiosensitivity parameters alpha (α) and beta (β) values. The RCS is predictive of clinical outcome.

Alternatively or additionally, the patient-specific radiosensitivity parameter alpha (α) value is predicted using a first machine learning model. Alternatively or additionally, the patient-specific radiosensitivity parameter beta (β) value is predicted using a second machine learning model. Optionally, the first and second machine learning models are support vector machines (SVMs).

Alternatively or additionally, the first set of signature genes includes at least one of HTRA1, C5orf17, KLHL6, DUSP27, FOS, PLCB4, WT1, PFN2, GNAI1, EVA1C, PIK3CG, ST8SIA6-AS1, and ATP8B4.

Alternatively or additionally, the second set of signature genes includes at least one of RRAGD, C5orf17, SOX8, CHRNA9, UMODL1, HOXC10, FGFBP1, HEMGN or EDAG, WT1, SCG5, CRYAB, GPX1, ZBED2, MAP2, RHAG, MSLN, and HSPA2.

Alternatively or additionally, the RCS function is based on a linear quadratic model for cell survival.

Alternatively or additionally, the subject is predicted to have a favorable clinical outcome when an RCS value is greater than a threshold.

Alternatively or additionally, the patient-specific dose and fractionation comprises a fraction dose and a number of fractions.

Alternatively or additionally, the tumor is a rectal, lung, or breast tumor.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 illustrates example operations for predicting personalized dose and fractionation for radiation therapy according to an implementation described herein.

FIG. 2 illustrates an example computing device.

FIG. 3 is a table listing genes for predicting patient-specific radiosensitivity parameter alpha (α) values.

FIG. 4 is a table listing genes for predicting patient-specific radiosensitivity parameter beta (β) values.

FIG. 5 is a model development process for RSIα/β according to an implementation described herein.

FIGS. 6A and 6B are graphs illustrating model development and initial validation for RSIα/β according to an implementation described herein.

FIGS. 7A-7C illustrate RSIα/β heterogeneity in 10,241 patients from the TCC by disease site according to an implementation described herein. FIG. 7D is a table illustrating the median α, β and α/β ration from the RSIα/β analysis within the TCC.

FIG. 8 is a table illustrating local control for validation in clinical cohorts according to an implementation described herein.

FIG. 9 is a hazard plot for a lung cohort according to an implementation described herein.

FIG. 10 is a table illustrating survival for validation according to an implementation described herein.

FIG. 11 is a hazard plot for a lung and glioma cohort according to an implementation described herein.

FIG. 12 is a hazard plot for a breast cohort according to an implementation described herein.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. While implementations will be described for predicting patient-specific dose and fractionation for radiation therapy to treat rectal, lung, and breast cancer, it will become evident to those skilled in the art that the implementations are not limited thereto, but are applicable for treating other types of tumors.

Described herein are systems and methods that use a machine learning algorithm for personalized radiation therapy. The algorithm uses patient-specific radiosensitivity parameters alpha (α) and beta (β) for a tumor and calculates a patient specific dose and fractionation using a radiation cytotoxicity score (RCS) function, where RCS is predictive of clinical outcome. The systems and methods described herein provide improvements over previously developed radiosensitivity index (RSI) and the genomic-adjusted dose (GARD) model to biologically-optimize radiotherapy (RT) dose, based on conventional fractionation (CF).

The patient specific radiosensitivity parameter alpha (α) and beta (β) for the tumor are calculated based on two independent sets of gene signatures using two independent machine learning model (RSIα/β). α is linearly related to dose while β is quadratically related to dose. The RT cytotoxicity score (RCS) function was derived using RSIα/β, the linear quadratic (LQ) model and each patient's dose/fractionation to predict the number of clones that were killed.

As described below (see Example 2), for the initial training, gene expression and survival curves of NCI60 cell lines were used to derive the model and it was further calibrated for heterogeneity across and within tumor types using 10,241 patients from the TCC by disease site. The algorithm was further validated with NCI60 cell lines and in clinical cohorts including 58 patients with stage III NSCLC treated with post-op RT, 596 breast cancer patients treated with surgery+/−RT and 44 anaplastic oligodendroglioma patients from EORTC 26951, a Phase 3 clinical trial. The results indicated that the systems and methods described herein predicted that a tumor with a lower α/β ratio is more sensitive to higher doses per fraction and patients with higher α/β ratio, are less sensitive to fraction size. This was further clinically validated in breast and lung TCC cohorts. For example, the breast validation cohort showed that the median RSIα/β ratio for patients achieving local control (LC) with conventional fractionation (CF) was higher than patients achieving local control with accelerated partial breast irradiation (APBI) (p<0.001). Further, there was no difference with α between the LC with APBI vs LC with CF (p=0.98), however a significant difference was found with β (p=0.03). This data further confirmed the treatment outcome prediction that patients with a lower RSIα/β ratio are more sensitive to fractionation and benefit from hypo-fractionated regimens.

The systems and methods described herein predict patient-specific values for radiosensitivity parameters α and β (also referred to herein as RSI_{α} and RSI_{β}) from genomic data. This is in contrast to conventional techniques that assume a broad estimate of the radiosensitivity parameters α and β (α/β=10). Additionally, as described herein, the systems and methods use independent machine learning models to predict RSI_{α} and RSI_{β}, respectively. With patient-specific values for radiosensitivity parameters, the systems and methods described herein can be used for total dose and fractionation optimization. This is not possible using conventional techniques. For example, the systems and methods described herein can identify patients who would benefit from total dose escalation and de-escalation compared to conventional techniques. Additionally, unique to this model it can identify patients who will benefit from hypo-fractionated radiation therapy (e.g., using the patient's α/β ratio). It should be understood that hypo-fractionated radiation therapy is delivered over a shorter period of time than conventional fractionation. Accordingly, the systems and methods described herein facilitate provision of personalized radiation therapy, which offers an improvement over the “one size fits all” approach used clinically.

The systems and methods described herein can be used to treat, or develop a treatment plan for, any solid tumor in a subject. A solid tumor is an abnormal mass of hyperproliferative or neoplastic cells from a tissue other than blood, bone marrow, or the lymphatic system, which may be benign or cancerous. In general, the tumors treated by the methods described herein are cancerous. As used herein, the terms “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of solid cancerous growths, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair. Examples of solid tumors are sarcomas, carcinomas, and lymphomas. Leukemias (cancers of the blood) generally do not form solid tumors.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some implementations, the disease is lung carcinoma, rectal carcinoma, colon carcinoma, esophageal carcinoma, prostate carcinoma, head and neck carcinoma, or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

In some implementations, the tumors treated by a method described herein are of epithelial cell origin. In some implementations, the tumors originate from rectal, lung, or breast tissues. In some implementations, the tumors originate from lung, colon, rectal, esophageal, prostate, or head/neck tissues (e.g., originating from the upper aerodigestive tract, including the lip, oral cavity, nasal cavity, paranasal sinuses, pharynx, and larynx, e.g., squamous cell carcinomas originating from the mucosal lining (epithelium)). In some implementations, the tumors are metastatic, and originate from an epithelial tissue (and are thus epithelial in origin) but have spread to another tissue, e.g., epithelial-origin prostate cancer that has spread to the bones of the pelvis, spine and/or ribs, or lung carcinoma that has metastasized to the adrenal glands, liver, brain, or bones.

A radiosensitivity index (RSI) and genomic-adjusted dose (GARD) as a model to biologically-optimize radiation therapy (RT) dose, based on conventional fractionation (CF), has been developed previously. For example, such techniques are described in detail in WO2016/179422, published Nov. 10, 2016, titled “SYSTEMS AND METHODS FOR PROVIDING PERSONALIZED RADIATION THERAPY.” RSI is a molecular estimate of the survival fraction at 2 Gy (“SF2”). RSI can be substituted for Survival in the standard linear quadratic model for cell survival as shown in Eqn. (1) below.

RSI=e^{-αD-βD\hat{ }2}, (1)

where α and ß are radiosensitivity parameters that provide measures of a tumor's ability to accumulate radiation damage, and D is the fraction dose (e.g., the radiation dose per treatment).

GARD is a subject-specific measure of the radiobiology parameter for dose effect shown in Eqn. (2) below.

GARD=nD(α+βD), (2)

where α and ß are radiosensitivity parameters that provide measures of a tumor's ability to accumulate radiation damage, D is the fraction dose (e.g., the radiation dose per treatment), and n is the number of radiation treatments (or fractions).

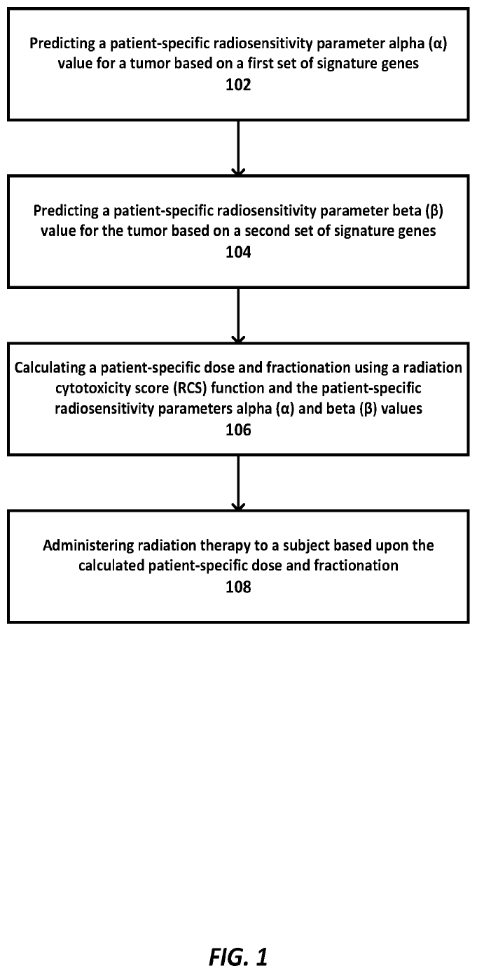

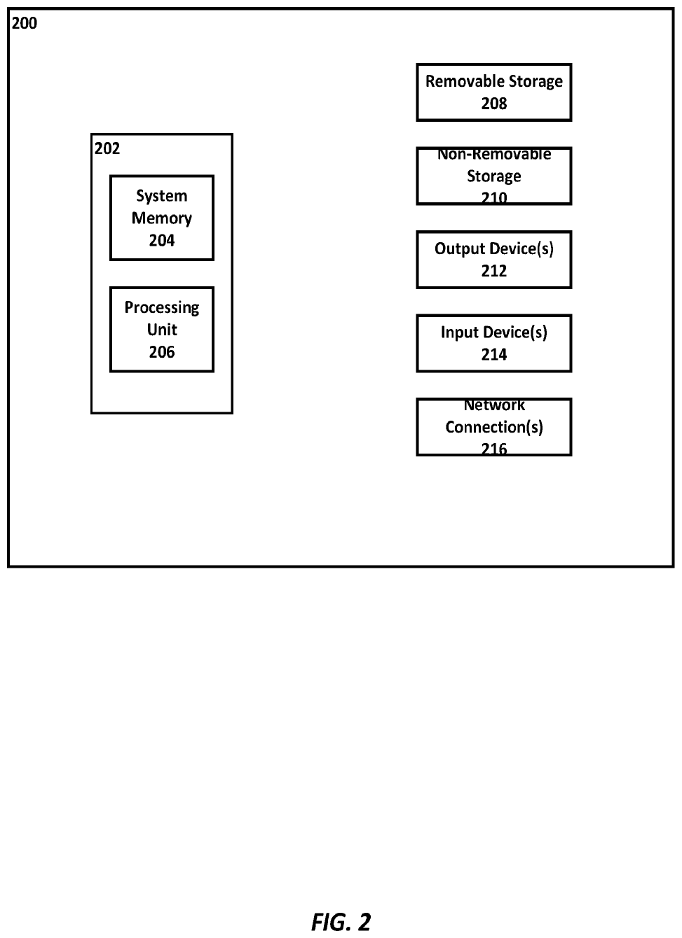

Referring now to FIG. 1, example operations for personalized dose and fractionation for radiation therapy are shown. This disclosure contemplates that the operations of FIG. 1 can be implemented using a computing device, for example, computing device 200 in FIG. 2. At step 102, a patient-specific radiosensitivity parameter alpha (α) (also referred to herein as “RSI_{α}”) value for a tumor is predicted. As described herein, the patient-specific radiosensitivity parameter alpha (α) value is predicted based on a first set of signature genes. The patient-specific radiosensitivity parameter alpha (α) value can be predicted using a first machine learning model. The first machine learning model is derived from gene expression and survival curves for radiotherapy doses (e.g., doses 2 Gy and 5 Gy). The first machine learning model predicts the patient-specific radiosensitivity parameter alpha (α) value directly from clonogenic assays on cell lines (e.g., NCI60). In some implementations described herein, the first machine learning model is a support vector machine (SVM). At step 104, a patient-specific radiosensitivity parameter beta (β) (also referred to herein as “RSI_{β}” or “β”) value for the tumor is predicted. As described herein, the patient-specific radiosensitivity parameter beta (β) value is predicted based on a second set of signature genes. The patient-specific radiosensitivity parameter beta (β) value can be predicted using a second machine learning model. The second machine learning model is derived from gene expression and survival curves for radiotherapy doses (e.g., doses 2 Gy and 5 Gy). The second machine learning model predicts the patient-specific radiosensitivity parameter beta (β) value directly from clonogenic assays on cell lines (e.g., NCI60). In some implementations described herein, the second machine learning model is an SVM. It should be understood that an SVM is provided only as an example machine learning model for predicting values for RSI_{α} and RSI_{β}. This disclosure contemplates using other machine learning algorithms including, but not limited to, artificial neural networks, classifiers (e.g., Naïve Bayes classifiers), random forests, regression techniques, etc. The tumor can optionally be a rectal, lung, or breast tumor. It should be understood that rectal, lung, and breast tumors are provided only as examples. This disclosure contemplates that the techniques described herein can be used for predicting values for RSI_{α} and RSI_{β} for other tumor types.

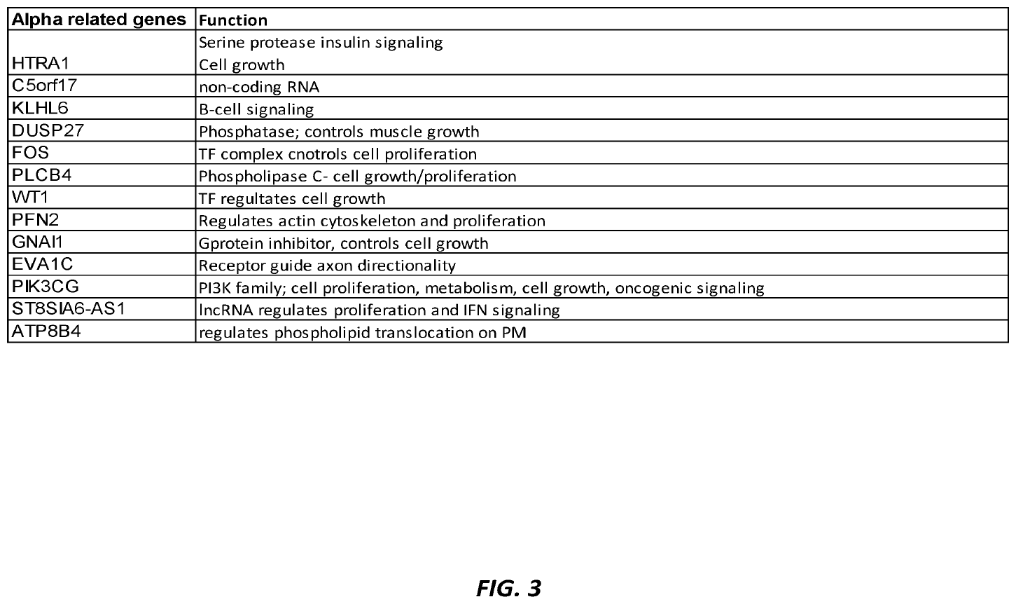

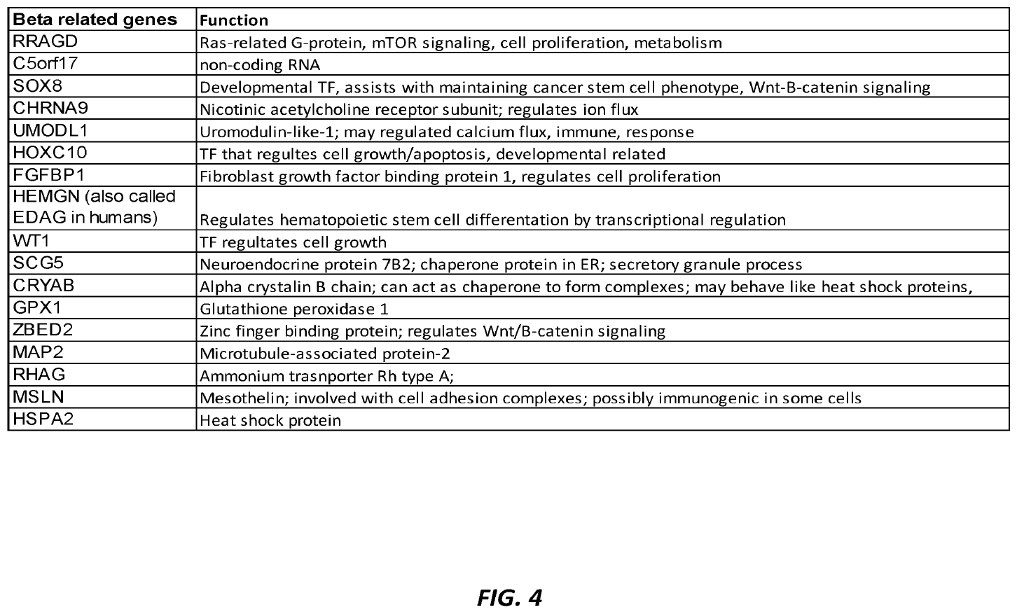

The patient-specific radiosensitivity parameters alpha (α) and beta (β) values can be predicted from expression levels of one or more signature genes of a cell or cells in the subject's tumor. The α model genes are listed in the table of FIG. 3 (i.e., at least one of HTRA1, C5orf17, KLHL6, DUSP27, FOS, PLCB4, WT1, PFN2, GNAI1, EVA1C, PIK3CG, ST8SIA6-AS1, and ATP8B4). The β model genes are listed in the table of FIG. 4 (i.e., at least one of RRAGD, C5orf17, SOX8, CHRNA9, UMODL1, HOXC10, FGFBP1, HEMGN or EDAG, WT1, SCG5, CRYAB, GPX1, ZBED2, MAP2, RHAG, MSLN, and HSPA2). One or more assays of cell(s) of the subject's tumor can be performed to determine gene expression levels. For example, any known technique for obtaining a sample comprising at least one living cell (preferably a plurality of cells), e.g., a cell from the subject's tumor (e.g., from a biopsy) can be used. Commonly used methods to obtain tumor cells include surgical (e.g., the use of tissue taken from the tumor after removal of all or part of the tumor) and needle biopsies. The samples should be treated in any way that preserves intact the gene expression levels of the living cells as much as possible, e.g., flash freezing or chemical fixation, e.g., formalin fixation. Additionally, any known technique can be used to extract material, e.g., protein or nucleic acid (e.g., mRNA) from the sample. For example, mechanical or enzymatic cell disruption can be used, followed by a solid phase method (e.g., using a column) or phenol-chloroform extraction, e.g., guanidinium thiocyanate-phenol-chloroform extraction of the RNA. A number of kits are commercially available for use in isolation of mRNA.

At step 106, a patient-specific dose and fractionation is calculated. The dose and fractionation can include a fraction dose (D) and a number of fractions (n). The patient-specific dose and fractionation is calculated using a radiation cytotoxicity score (RCS) function, which is based on a linear quadratic model for cell survival. The RCS function is shown in Eqn. (3) below.

RCS=(e^{(-RSI}^{_{α}}^{*D-RSI}^{_{β}}^{*D}^{²}⁾)ⁿ, (3)

where RSI_{α} is the radiosensitivity parameter alpha (α), RSI_{ß} is the radiosensitivity parameter beta (ß), D is the fraction dose, and n is the number of fractions. The patient-specific values for RSI_{α} and RSI_{ß} predicted by steps 102 and 104, respectively, are used to calculate the patient-specific dose and fractionation. RCS is predictive of clinical outcome. For example, the patient is predicted to have a favorable clinical outcome if the RCS value is greater than a threshold. This disclosure contemplates that the RCS threshold values are particular to tumor type. It should be understood that fraction dose (D) and a number of fractions (n) can be derived for known values of RCS (e.g., a value above the threshold), RSI_{α} and RSI_{ß}. Optionally, at step 108, radiation therapy is administered to a subject using the patient-specific dose and fractionation, e.g., with the fraction dose (D) and number of fractions (n) calculated at step 106. The operations of FIG. 1 can be used to identify subjects whose tumors may benefit from hypo-fractionated radiation therapy. Thus, radiation therapy delivered at step 108 can be a hypo-fractionated regimen. Alternatively or additionally, the operations of FIG. 1 can be used to identify subjects whose tumors may benefit from total dose escalation or de-escalation.

It should be appreciated that the logical operations described herein with respect to the various figures may be implemented (1) as a sequence of computer implemented acts or program modules (i.e., software) running on a computing device (e.g., the computing device described in FIG. 2), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 2, an example computing device 200 upon which the methods described herein may be implemented is illustrated. It should be understood that the example computing device 200 is only one example of a suitable computing environment upon which the methods described herein may be implemented. Optionally, the computing device 200 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 200 typically includes at least one processing unit 206 and system memory 204. Depending on the exact configuration and type of computing device, system memory 204 may be volatile (such as random access memory (RAM)), non-volatile (such as read-only memory (ROM), flash memory, etc.), or some combination of the two. This most basic configuration is illustrated in FIG. 2 by dashed line 202. The processing unit 206 may be a standard programmable processor that performs arithmetic and logic operations necessary for operation of the computing device 200. The computing device 200 may also include a bus or other communication mechanism for communicating information among various components of the computing device 200.

Computing device 200 may have additional features/functionality. For example, computing device 200 may include additional storage such as removable storage 208 and non-removable storage 210 including, but not limited to, magnetic or optical disks or tapes. Computing device 200 may also contain network connection(s) 216 that allow the device to communicate with other devices. Computing device 200 may also have input device(s) 214 such as a keyboard, mouse, touch screen, etc. Output device(s) 212 such as a display, speakers, printer, etc. may also be included. The additional devices may be connected to the bus in order to facilitate communication of data among the components of the computing device 200. All these devices are well known in the art and need not be discussed at length here.

The processing unit 206 may be configured to execute program code encoded in tangible, computer-readable media. Tangible, computer-readable media refers to any media that is capable of providing data that causes the computing device 200 (i.e., a machine) to operate in a particular fashion. Various computer-readable media may be utilized to provide instructions to the processing unit 206 for execution. Example tangible, computer-readable media may include, but is not limited to, volatile media, non-volatile media, removable media and non-removable media implemented in any method or technology for storage of information such as computer readable instructions, data structures, program modules or other data. System memory 204, removable storage 208, and non-removable storage 210 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 206 may execute program code stored in the system memory 204. For example, the bus may carry data to the system memory 204, from which the processing unit 206 receives and executes instructions. The data received by the system memory 204 may optionally be stored on the removable storage 208 or the non-removable storage 210 before or after execution by the processing unit 206.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### EXAMPLES

### Example 1

The systems and methods described herein aim to develop a new model to biologically-optimize fractionation.

Materials/Methods:

Public data for the NCI60 cancer cell lines, including gene expression (GE, Affymetrix U133 2.0) and survival curves was utilized. Alpha (α) and beta (β) were calculated based on the linear quadratic model (LQ). Two machine learning models to predict α and β (RSIα/β) were developed using GE and support vector machines using six-fold cross validation. RT cytotoxicity score (RCS) was calculated using RSIα/β, the LQ and each patient's dose/fractionation to predict the number of clones killed. RCS was calibrated in 58 patients with stage III NSCLC treated with post-op RT and thresholds that distinguished clinical outcome were identified. The locked-down RCS was tested in two independent cohorts, including 596 breast cancer patients treated with surgery+/−RT (median f/u=8 yrs) and 44 anaplastic oligodendroglioma (AO) patients from EORTC 26951, a Phase 3 clinical trial. In the breast cohort, patients received RT with CF (n=336, median 60 Gy), accelerated partial breast RT (n=75 APBI, median 34 Gy) or no RT (n=260). In AO patients received 59.4 Gy. The lung/breast cohorts were from our IRB-approved biorepository. GE was from Affymetrix Hu-RSTA (breast, lung) and U133 Plus (AO). Time to event analysis was with Kaplan-Meier estimates and log-rank test. RCS and outcomes were explored with univariable and multi-variable (MVA) Cox regression.

Results:

In the calibration cohort (NSCLC), patients with a predicted optimized RCS had an improved 1-yr local control (LC)(84.3% vs 57.1% p<0.001) and overall survival (OS)(4.8 vs 1.9 yrs p=0.04). In patients with ≥R1 resection, a predicted a higher RCS showed improved 1-yr LC (91% vs 69.2% p=0.005) and OS (5.1 vs 2.6 yrs p=0.049). In EORTC 26951 patients that achieved an optimized RCS had superior OS (5.7 vs 2.6 yrs p=0.02). In the RT breast cohort, patients who achieved an optimized RCS had superior 8 yr LC (91% vs 72% p=0.03) that is significant on MVA (HR 0.29, p<0.014). In non-RT patients, RCS (generated by simulating CF (50 Gy)) did not predict 8 yr LC (p=0.95). The median α/β ratio of locally controlled patients with APBI was lower than CF (5.5 vs 7.9 p<0.001).

Conclusion:

As described herein, a clinically-actionable machine learning model that predicts RCS, an individualized RT cytotoxicity score has been developed and validated. RCS predicts no fractionation approach is universally optimal and provides a basis to biologically-tailor RT dose and fractionation.

### Example 2

Described below is a new model, RSIα/β, trained on the NCI60 from the ground up to derive a genomic expression estimates of α and β of the LQ model. Unlike RSI, RSIα/β was trained to predict α and β values directly from clonogenic assays on NCI60 rather than indirectly (RSI->GARD) utilizing machine learning. With β now modeled, RSIα/β can be used to investigate fractionation optimization. Like RSI, RSIα/β I used to develop GARDα/β to predict response to radiation dose prescriptions by utilizing the LQ model. Thus, emphasizing that the “one size fits all” approach is flawed as it applies to the total dose in addition to fraction size.

Materials and Methods:

Calculating α and β from NCI60 clonogenic assays: Cellular survival curves for radiotherapy doses of 2 and 5 Gy, were publicly-available for the NCI panel of 60 cancer cell lines (NCI60). The α and β parameters were calculated for each cell lines using the linear quadratic model:

SF=e^{-αD-βD\hat{ }2}.

Gene expression data (Affymetrix U-133 Plus 2.0) were obtained from CellMiner (https://discover.nci.nih.gov/cellminer/). Raw gene expression data were extracted from the .cel files. The gene-level expression values were normalized with Robust Multi-array Average (RMA) using RMAExpress software version 1.1.0. The probeset-level expression values were converted to gene-level. Multiple probesets in the same gene were merged together using their geometric mean. Common genes covered by both the Affymetrix U-133 2.0 plus platform and the custom Affymetrix Hu-RSTA-2a520709 were identified (18,514 genes) for subsequent cross-platform analyses. Data pre-processing and preparation were achieved using Python version 3.5.3 with custom scripts.

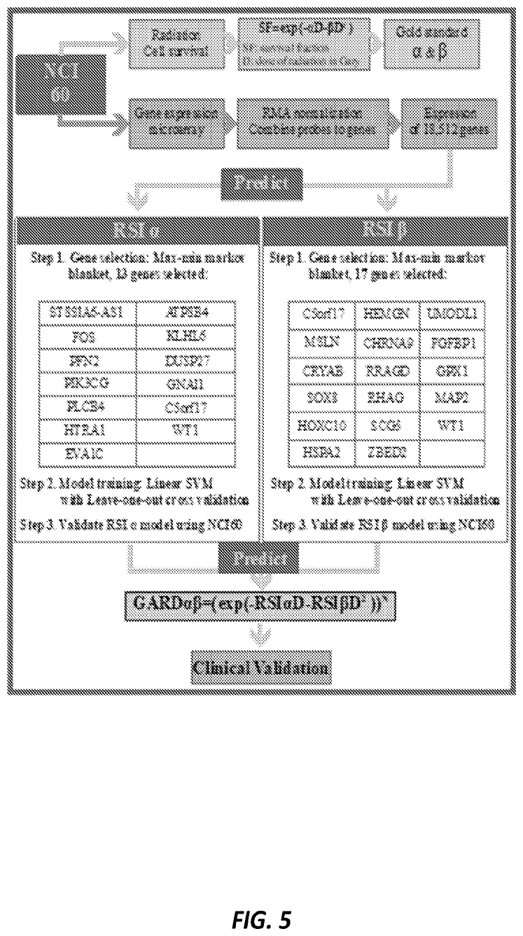

RSI α and β model development: Machine learning (support vector machine, SVM) was used to develop two independent gene expression-based models to predict α and β. The α and β values calculated based on NCI60 clonogenic data were used as gold-standard values to train the models (FIG. 5). Briefly, 18,512 common genes were first identified between the two Affymetrix platforms. Then, Max-Min Markov Blanket method was used to reduce the number of genes through a forward-backward search using R package MXM. The output non-redundant set of genes for each model was used to construct RSIα/β models using R package e1071. The performance of the model was evaluated using 10-fold cross validation.

Validation RSIα/β model (cell lines): Predicted α and β generated from RSIα/β were compared against α and β values derived from clonogenic assays of the NCI60 using Pearson Correlation coefficients. The model outputs were used to calculate predicted SF2 and SF5 based on the LQ model. Correlations between the predicted and observed SF2 and SF5 from clonogenic assays were calculated using Pearson Correlation coefficients. All these correlation measurements were calculated using R.

GARDα/β development: GARDα/β is derived from GARD with an individualized β parameter. Briefly, GARD is a metric to estimate the efficacy of radiation based on the LQ model and genomic expression. Patient specific α is derived from RSI to calculate GARD with a β assumption of 0.05. Unlike GARD, GARDα/β does not require a β assumption since α and β are derived from RSIα/β.

GARD_{α/ß}=n*D(RSI_{α}+RSI_{ß}*D),

where RSI_{α} is the patient-specific radiosensitivity parameter alpha (α), RSI_{ß} is the patient-specific radiosensitivity parameter beta (ß), D is the fraction dose, and n is the number of fractions.

Clinical Cohorts:

TCC cohorts: The RSIα/β distributions from 10,271 patients across all disease sites were acquired from the Total Cancer Care (TCC), a prospective IRB-approved data and tissue collection protocol active at H. Lee Moffitt Cancer Center and Research Institute (“Moffitt”) and 18 other institutions since 2006. Tumors from patients enrolled in the TCC protocol were profiled on the Affymetrix Hu-RSTA-2a520709 chip (Affymetrix, Santa Clara, CA), which contains approximately 60,000 probe sets representing 25,000 genes. All patients in the TCC cohort were consented for the TCC protocol.

MCC Lung and breast cancer cohort: These cohorts were derived from the TCC with retrospectively collected clinical outcomes. The lung cohort includes a total of 58 patients with stage III NSCLC treated at Moffitt with post-operative RT (dose range 45-70 Gy). The median follow up was 59.5 months. The breast cohort includes a total of 671 breast cancer patients treated with surgery+/−RT (dose range 34-66 Gy) with a median follow up of 8 years. In the breast cohort, patients received RT with conventional fractionation (n=336, median 60 Gy), accelerated partial breast RT (APBI) (n=75, median dose 34 Gy) or no RT (n=260). The clinical end point was local control and overall survival for both cohorts.

EORTC Glioma (GSE43388): This cohort contains 140 patient tumor samples from the EORTC26951 Phase III clinical trial on Anaplastic Oligodendroglioma (glioma), 42 were profiled on the Affymetrix U133 Plus 2.0 chip and were used for analysis. The .cel files were obtained from GEO. The median follow-up was 140 months and all patients were treated with 59.4 Gy in 1.8 Gy per fraction+/−temozolomide. The available clinical end point was overall survival. (See van den Bent M J, Brandes A A, Taphoorn M J B, et al. Adjuvant procarbazine, lomustine, and vincristine chemotherapy in newly diagnosed anaplastic oligodendroglioma: long-term follow-up of EORTC brain tumor group study 26951. J Clin Oncol Off J Am Soc Clin Oncol. 2013; 31(3):344-350).

All of the raw .cel files were normalized utilizing RMAExpress 1.1.0.

Statistics:

Previous work demonstrated the ability to perform multi-cohort analysis for RSI and GARD. The same analysis was performed for GARDα/β in the clinical cohorts.

Cox regression model was used to model the relationship between clinical outcome and GARDα/β. The proportional-hazards assumption was tested, and it holds for all covariates in the model. In order to statistically compare cohorts which are heterogeneous in outcome and disease type, separate models were built for patients who received and did not receive XRT. Specifically, a model was built for each outcome/disease combination. This allowed each to have a different baseline hazard function yet still estimate a common GARDα/β effect. Models were built separately depending whether or not patients received XRT. Restricted cubic splines, with 5 knots, were utilized to allow for non-linear effects of GARDα/β in each stratified Cox model. There was, however, no evidence of non-linear effects for GARDα/β for any outcome. For simplicity then, the results were restricted to linear modeling.

Including a continuous variable analysis across all three cohorts, a historically significant cytotoxic threshold was used to dichotomize patients. Jack Fowler in 2007 estimated an ^{˜}11 log 10 cell kill in all the most effective H&N RT schedules. 11 log 10 cytotoxicity reduces surviving cells from approximately 10⁹ cells per gram of tissue to a chance of one cell in 100 tumors surviving radiation assuming no repopulation. (See Pretreatment Transcriptional Profiling for Predicting Response to Neoadjuvant Chemoradiotherapy in Rectal Adenocarcinoma|Clinical Cancer Research. https://clincancerres.aacrjournals.org/content/17/9/3039. Accessed Dec. 5, 2019). 11 log 10 cytotoxicity is equivalent to a GARDα/β value of 22 (GARDα/β22). To validate this cut point, patients were dichotomized with a GARDα/β22 threshold. Multivariate cox proportional hazards regression to assess the association between GARDα/β22 and the clinical endpoint, adjusting for potential confounders.

Results:

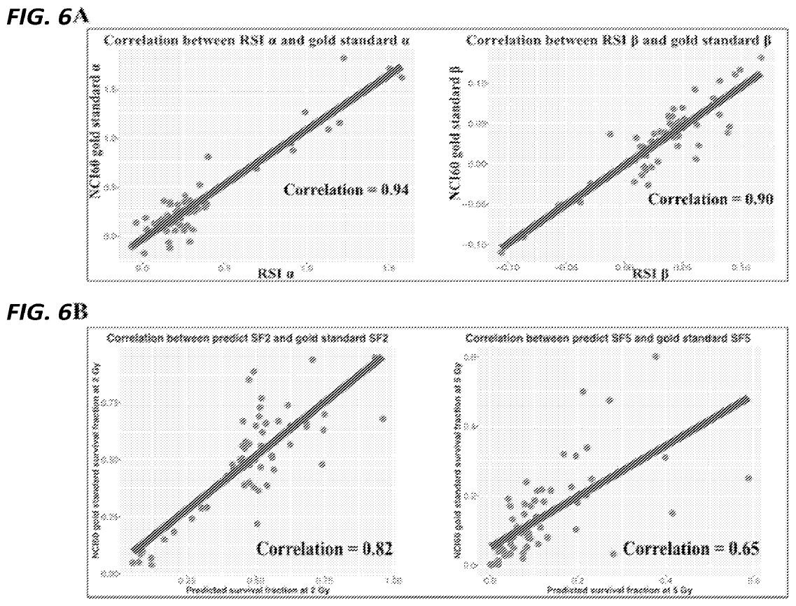

FIGS. 6A and 6B demonstrates model development and initial validation for RSIα/β. Following the Max-Min Markov Blanket feature selection step, 13 and 17 genes were selected for α and β model, respectively. There are two gene similarities found in RSIa and RSIb: C5orf17 and WT1. The genes in both models are mechanistically involved in cell adhesion, proliferation and have been reported to be directly associated with various cancers (Supplemental Figure pathway analysis). The ability of RSIα/β to reconstruct clonogenic assay derived α and β from the NCI60 (Figure x). There is a high correlation between the RSIα/β and observed values were observed for both models (Pearson's R=0.94 and 0.90 for α and β, respectively). Predicted SF2 and SF5 were calculated using RSIα/β and the LQ equation and compared with the actual SF2 and SF5 (Pearson's R=0.82 and 0.65 for α and β, respectively). These results indicate that the RSIα/β is informative in predicting and reconstructing the cellular fate of NCI60 samples.

Distribution of RSIα/ß Across Disease Sites:

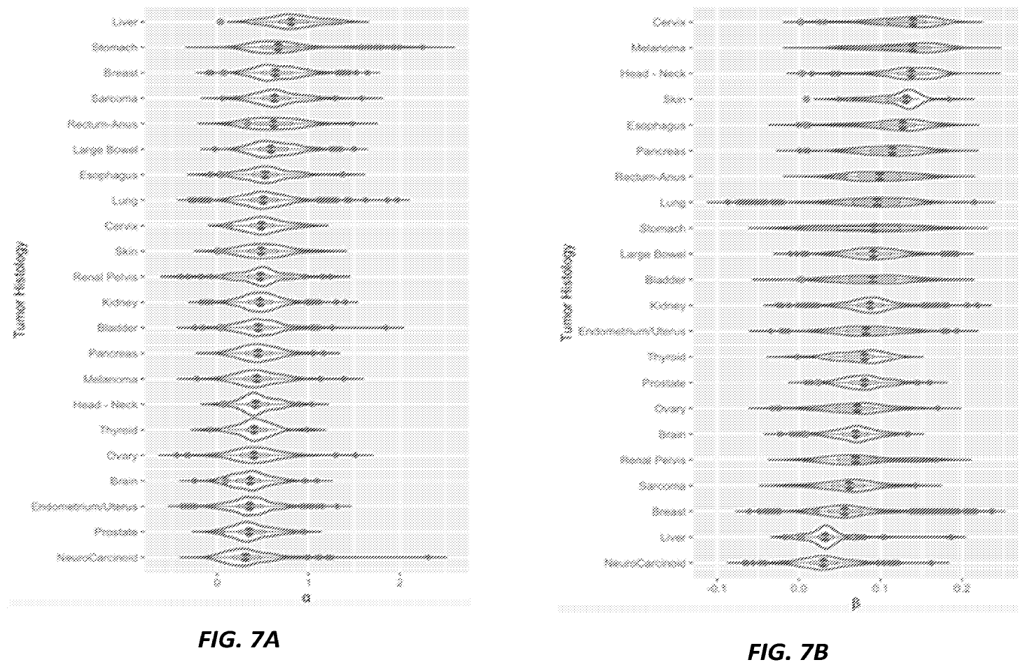

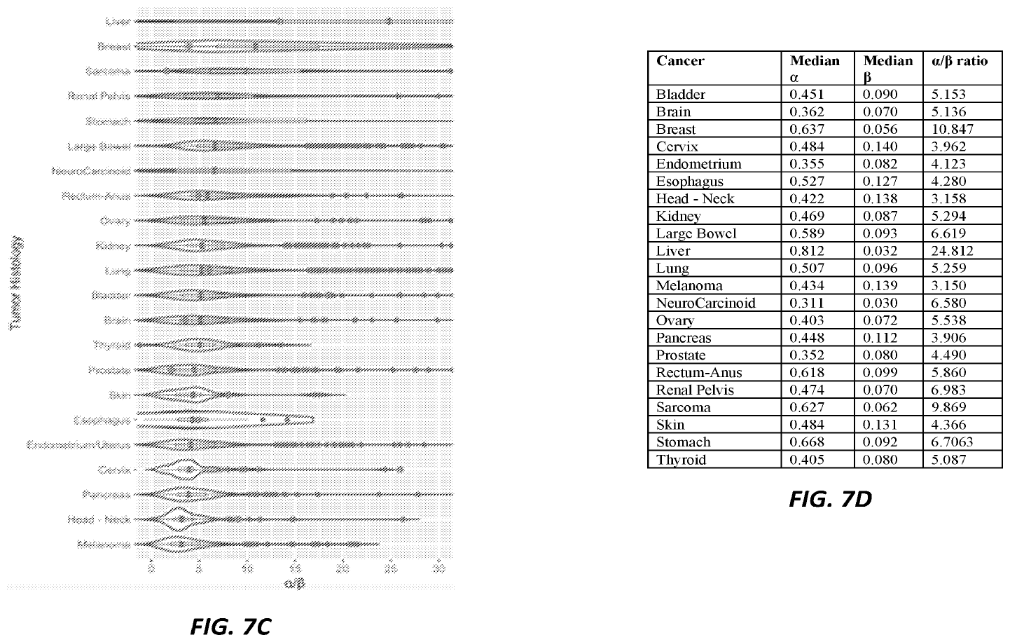

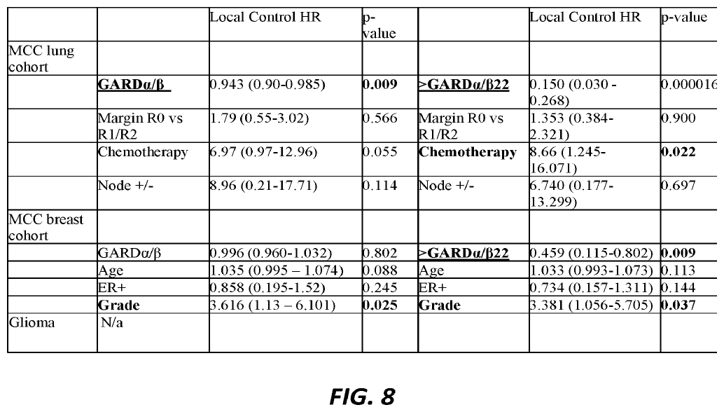

FIGS. 7A-7C illustrate RSIα/β heterogeneity in 10,241 patients from the TCC by disease site. The red dot denotes medians α and β values across disease sites from a study of 64 different studies. (See van Leeuwen C M, Oei A L, Crezee J, et al. The alfa and β of tumours: a review of parameters of the linear-quadratic model, derived from clinical radiotherapy studies. Radiat Oncol Lond Engl. 2018; 13. doi:10.1186/s13014-018-1040-z). In FIG. 7C, the x axis was restricted to 0-30 in order to include clinically meaningful α/β ratios. This omitted some data in some disease sites where extremely low beta values would yield extremely large α/β ratios. FIG. 7D is a table illustrating the median α, β, and α/β ratio from the RSIα/β analysis within the TCC.

In RSIα/β, there is a large distribution across and within tumor types. Most apparent is the common assumption of a tumor α/β ratio of 10 is often not accurate. In fact, the heterogeneity in β, which is quadratically related to dose, reveals certain tumor types benefit from hypofractionation more than others. For example, three tumor types with the largest β values include melanoma, non-melanoma skin cancer, and cervical cancer are consistent with extensive clinical data supporting the advantages of hypofractionation radiation in the treatment of these cancers. (See Strojan P. Role of radiotherapy in melanoma management. Radiol Oncol. 2010; 44(1):1-12. doi:10.2478/v10019-010-0008-x; Gunaratne D A, Veness M J. Efficacy of hypofractionated radiotherapy in patients with non-melanoma skin cancer: Results of a systematic review. J Med Imaging Radiat Oncol. 2018; 62(3):401-411. doi:10.1111/1754-9485.12718). However, within every tumor type there are patients that will benefit from hypofractionation more than others.

Of note, within the distribution there are a small portion of patients that have negative α and β values. Negative α and β values are found in previous studies with estimates of α and β parameters from clinical data. (See van Leeuwen C M, Oei A L, Crezee J, et al. The alfa and β of tumours: a review of parameters of the linear-quadratic model, derived from clinical radiotherapy studies. Radiat Oncol Lond Engl. 2018; 13. doi:10.1186/s13014-018-1040-z). These negative samples likely do not fit the LQ model.

GARDα/ß Validation in Clinical Cohorts:

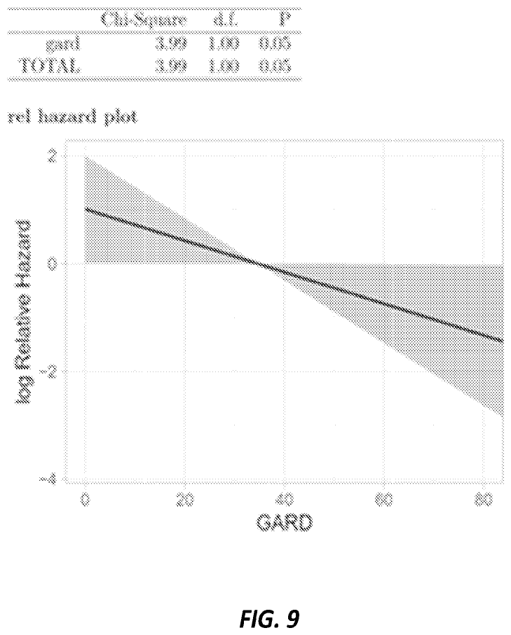

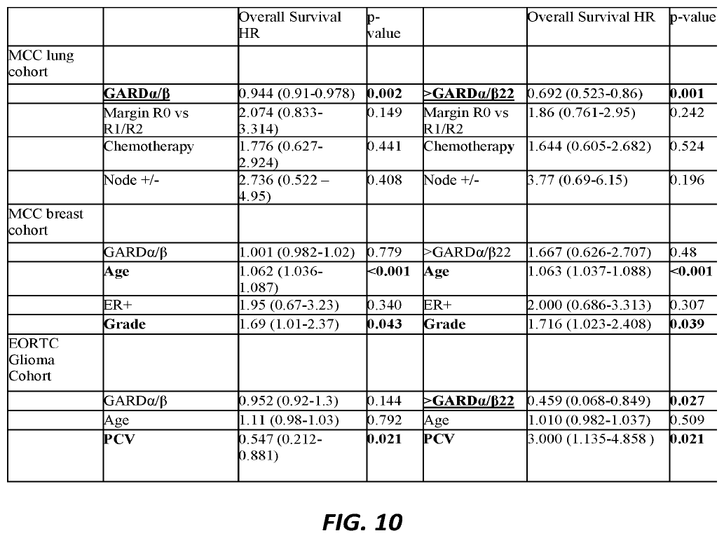

As shown in FIG. 9, >GARDα/β22 shows superior local control in the lung (HR 0.15 (0.03−0.268) p<0.001) and breast (HR 0.459 (0.12−0.80 p=0.009) on multivariate analysis. As a continuous variable within the lung cohort GARDα/β is significant (HR 0.94 (0.90−0.985) p=0.009), but not in the breast cohort (p=0.802). While considering both cohorts together GARDα/β is significant as a continuous variable (p=0.05).

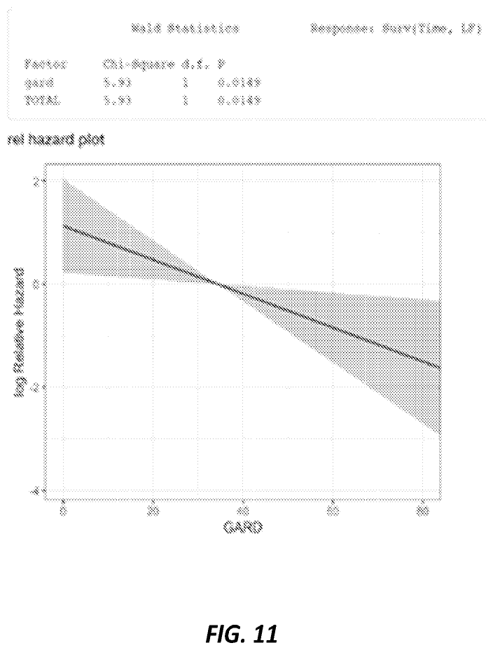

As shown in FIG. 11, in the lung and glioma cohort, >GARD α/β22 shows superior OS (HR 0.94 (0.90−0.985) p=0.009) on multivariate analysis. As a continuous variable within the lung cohort GARDα/β is significant, but not in the breast or glioma cohort. While considering all cohorts together GARDα/β is significant continuously (p=0.0149).

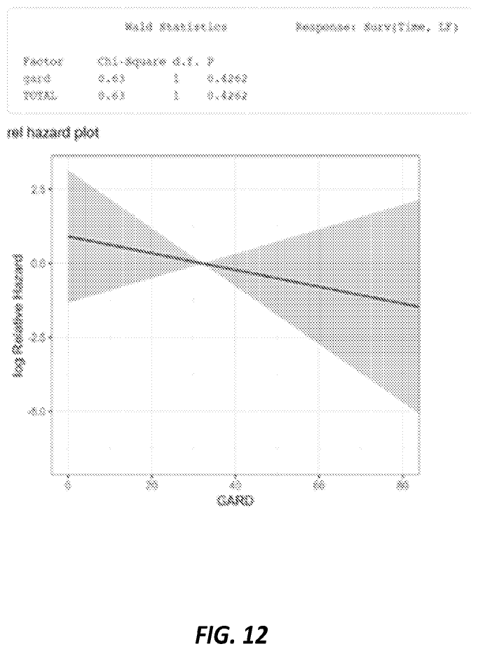

As shown in FIG. 12, GARDα/β does not predict for outcomes of breast cancer patients (n=260 p=0.426) treated with surgery alone (50 Gy in 25 fractions used to calculate GARDα/β), suggesting the RT-specificity of the model.

Hypofractionation Benefits Lower α/ß Ratio in the Breast Cohort:

According to the LQ model, a tumor with a lower α/β ratio is more sensitive to higher doses per fraction. These patients have a larger shoulder at conventional RT doses on their survival curve and therefore require a higher dose to achieve quadratic killing. Conversely, patients with higher α/β ratio experience less of a shoulder and are less sensitive to fraction size.

To determine whether α/β ratio differences are of clinical relevance, this concept was tested in the breast cohort were patients were treated with either standard fractionation (n=250) or APBI (n=75). The median RSIα/β ratio for patients achieving local control with conventional fractionation (LC CF) is higher than patients achieving local control with APBI (LC APBI 3.4 Gy/fraction) (19.51 vs 10.3, p<0.001). There is no difference in α between the LC APBI vs LC CF (0.526 vs 0.530, p=0.98) but there is a significant difference between β (0.05 vs 0.027, p=0.03). This data suggests that patients with a lower RSIα/β ratio are more sensitive to fractionation and benefit from a hypofractionated regimens.

Discussion:

RSIα/β extends on the previous work developed in RSI by estimating α and β parameters of the LQ model based on genomic expression. NCI60 was chosen for model training in order to predict intrinsic radiosensitivity in a histological agnostic manner. Similar to RSI and the LQ model itself, unique histological biology is captured within the estimates of α and β. Therefore, any tumors with the same RSIα/β will have the same probability of control given the same dose and fraction size. This could have a large impact on future clinical trial designs. Rather than empirically assigning dose and fractionation schema within specific disease sites, one can use RSIα/β and GARDα/β to design biologically optimized radiation prescriptions across any tumor based on genomic expression.

Despite the usefulness of the LQ model in clinical practice, there is little consensus of α and β values within tumor types. For example, in a study that included an analysis of 64 studies (See van Leeuwen C M, Oei A L, Crezee J, et al. The alfa and β of tumours: a review of parameters of the linear-quadratic model, derived from clinical radiotherapy studies. Radiat Oncol Lond Engl. 2018; 13. doi:10.1186/s13014-018-1040-z) estimating α and β across different diseases sites showed an α/β ratio range of −59.4-30 in head and neck tumors. Several values in the analysis of RSIα/β across all disease sites are consistent with α and β values reported in the literature, however other values such as bladder and cervical estimates are significantly different. Previous studies that estimate α and β within histology allow for only broad estimates of radiosensitivity parameters in the pre-treatment setting. The example described herein the first known model to estimate both α and β from non-histological pre-treatment information to prospectively individualize response to radiotherapy.

GARD was the first radiosensitivity metric that quantitatively modifiable based RT dose. GARDα/β takes an additional step forward by personalizing fraction size with the β parameter to determine sensitivity to radiation treatment. Fraction size optimization is increasingly relevant with clinical data showing hypofractionation and SBRT offers superior outcomes, less overall treatment time and patient burden compared to conventional fractionation. Prostate, lung and pancreas are few examples where hypofractionation superiority over conventional fractionation in clinical trials are emerging. This is consistent in the RSIα/β analysis where these tumor types have an α/β ratio lower than the broad estimation of 10. Furthermore, by personalizing α and β there are patients within all tumor types that would benefit from hypofractionation, thus emphasizing a histological agnostic approach to radiotherapy.

This example demonstrates the effectiveness of a given radiation schedule is biologically distinct between patients and is probabilistic in nature. GARDα/β can be used to quantify the probability of disease control based on cytotoxicity as a continuous variable in the pooled cohort analysis. As a continuous variable the probability of tumor eradication increases as GARD α/β increases, and vice versa. Within each cohort individually GARDα/β is only significant as a continuous variable in the lung cohort for both local control and survival.

Of note, since GARDα/β is a measure of cytotoxicity, initial tumor burden is not considered at this time. In 2007, Jack Fowler estimated a 11 log 10 (GARDα/β22) threshold for all the most effective H&N radiotherapy schedules. 11 log 10 cytotoxicity assumes a 1% chance the tumor will not be eliminated in a gram of tissue. This example validates this cut point across all three cohorts. However, when dichotomizing patients utilizing the GARDα/β threshold that yields the maximum chi-square value within each cohort, one can estimate the initial tumor burden by keeping the aforementioned probability of control constant. This estimates an initial tumor volume of 10 mg, 100 mg, and 1 g in breast, lung and glioma, respectively. This trend correlates well with the percent of non R0 (in lung and breast) and non-gross total resections (glioma) within each cohort breast (5%), lung (41%), and glioma (59%). Future work will involve integrating initial tumor burden to stratify probability of risk of control with radiation.

Today, the LQ model is commonly used to calculate isoeffective doses utilizing the α/β ratio (biological effective dose (BED) and equivalent dose delivered in 2 Gy fractions (EQD2)). These equations are formulations of the LQ equation—calculating isoeffective doses within tumor with equivalent α and β.

$$BED = nD ( 1 + \frac{D}{( α / β )} ) ,$$

where α and ß are radiosensitivity parameters that provide measures of a tumor's ability to accumulate radiation damage, D is the fraction dose, and n is the number of fractions.

Therefore, it is important to consider the values of α and β and not simply the α/β ratio. For example, GARDα/β will vary significantly for two tumors with an a=0.5 and b=0.05 vs a=1 and b=0.1 despite having the same α/β ratio of 10. BED and EQD2 are frequently used across different tumors within histology using same α/β ratio with the assumption intrahistological tumors have similar α and β. With RSIα/β, the intrahistological heterogeneity is quantified and uncovers the importance of considering values of α and β. Indeed, in the prior example of the two tumors with the same α/β ratio but differing α and β, both BED and EQD2 formulas would not yield isoffective doses.

Like GARD, GARDα/β does not consider normal tissue toxicity and the therapeutic ratio. It is currently a platform to optimize tumor eradication. It should be understood that the importance of fractionation schema is limited to normal and not tumor tissue tolerance. Indeed, it is hypothesized the advantage of conventional fractionation is the sparing late responding tissue assumed to have a lower α/β ratio. Still, many clinical trials have shown similar rates of toxicity between hypofractionated and conventional regimens.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A method for personalized radiation therapy, comprising:
predicting a patient-specific radiosensitivity parameter alpha (α) value for a tumor, the patient-specific radiosensitivity parameter alpha (α) value being predicted based on a first set of signature genes;
predicting a patient-specific radiosensitivity parameter beta (β) value for the tumor, the patient-specific radiosensitivity parameter beta (β) value being predicted based on a second set of signature genes; and
calculating a patient-specific dose and fractionation using a radiation cytotoxicity score (RCS) function and the patient-specific radiosensitivity parameters alpha (α) and beta (β) values, wherein RCS is predictive of clinical outcome.

2. The method of claim 1, further comprising administering radiation therapy to a subject based upon the calculated patient-specific dose and fractionation.

3. The method of claim 2, wherein the administered radiation therapy is a hypo-fractionated radiation therapy regimen.

4. The method of claim 1, wherein the patient-specific radiosensitivity parameter alpha (α) value is predicted using a first machine learning model, and wherein the patient-specific radiosensitivity parameter beta (β) value is predicted using a second machine learning model.

5. The method of claim 4, wherein the first and second machine learning models are support vector machines (SVMs).

6. The method of claim 1, wherein the first set of signature genes comprises at least one of HTRA1, C5orf17, KLHL6, DUSP27, FOS, PLCB4, WT1, PFN2, GNAI1, EVA1C, PIK3CG, ST8SIA6-AS1, and ATP8B4.

7. The method of claim 1, wherein the second set of signature genes comprises at least one of RRAGD, C5orf17, SOX8, CHRNA9, UMODL1, HOXC10, FGFBP1, HEMGN or EDAG, WT1, SCG5, CRYAB, GPX1, ZBED2, MAP2, RHAG, MSLN, and HSPA2.

8. The method of claim 1, wherein the RCS function is based on a linear quadratic model for cell survival.

9. The method of claim 1, wherein the subject is predicted to have a favorable clinical outcome when an RCS value is greater than a threshold.

10. The method of claim 1, wherein the patient-specific dose and fractionation comprises a fraction dose and a number of fractions.

11. The method of claim 1, wherein the tumor is a rectal, lung, or breast tumor.

12. A system for personalized radiation therapy, comprising:
a processor; and
a memory operably coupled to the processor, the memory having computer-executable instructions stored thereon that, when executed by the processor, cause the processor to:
predict a patient-specific radiosensitivity parameter alpha (α) value for a tumor, the patient-specific radiosensitivity parameter alpha (α) value being predicted based on a first set of signature genes;
predict a patient-specific radiosensitivity parameter beta (β) value for the tumor, the patient-specific radiosensitivity parameter beta (β) value being predicted based on a second set of signature genes; and
calculate a patient-specific dose and fractionation using a radiation cytotoxicity score (RCS) function and the patient-specific radiosensitivity parameters alpha (α) and beta (β) values, wherein RCS is predictive of clinical outcome.

13. The system of claim 12, wherein the patient-specific radiosensitivity parameter alpha (α) value is predicted using a first machine learning model, and wherein the patient-specific radiosensitivity parameter beta (β) value is predicted using a second machine learning model.

14. The system of claim 13, wherein the first and second machine learning models are support vector machines (SVMs).

15. The system of claim 12, wherein the first set of signature genes comprises at least one of HTRA1, C5orf17, KLHL6, DUSP27, FOS, PLCB4, WT1, PFN2, GNAI1, EVA1C, PIK3CG, ST8SIA6-AS1, and ATP8B4.

16. The system of claim 12, wherein the second set of signature genes comprises at least one of RRAGD, C5orf17, SOX8, CHRNA9, UMODL1, HOXC10, FGFBP1, HEMGN or EDAG, WT1, SCG5, CRYAB, GPX1, ZBED2, MAP2, RHAG, MSLN, and HSPA2.

17. The system of claim 12, wherein the RCS function is based on a linear quadratic model for cell survival.

18. The system of claim 12, wherein the subject is predicted to have a favorable clinical outcome when an RCS value is greater than a threshold.

19. The system of claim 12, wherein the patient-specific dose and fractionation comprises a fraction dose and a number of fractions.

20. The system claim 12, wherein the tumor is a rectal, lung, or breast tumor.
